(19) United States
(12) Patent Application Publication
(10) Pub. No.: US 2025/0114388 A1
(43) Pub. Date: Apr. 10, 2025

(54) ACTIVE PHARMACEUTICAL INGREDIENT COMPOSITIONS AND PREPARATION AND USES THEREOF

(71) Applicant: Dr. Anti Fungus, LLC, Oxnard, CA (US)

(72) Inventors: Andrew Langroudi, Oxnard, CA (US); Nasim Kalhor, Oxnard, CA (US)

(21) Appl. No.: 18/910,649

(22) Filed: Oct. 9, 2024

Related U.S. Application Data
(60) Provisional application No. 63/588,795, filed on Oct. 9, 2023.
(60) Provisional application No. 63/599,949, filed on Nov. 16, 2023.
(60) Provisional application No. 63/670,307, filed on Jul. 12, 2024.

(51) Int. Cl. A61K 31/7048; A61K 31/137; A61K 31/201; A61K 31/27; A61K 31/4174; A61K 31/4196; A61K 31/496; A61K 38/12; A61K 47/10; A61K 47/14; A61K 47/26; A61P 31/10

(52) U.S. Cl. CPC A61K 31/7048; A61K 31/137; A61K 31/201; A61K 31/27; A61K 31/4174; A61K 31/4196; A61K 31/496; A61K 38/12; A61K 47/10; A61K 47/14; A61K 47/26; A61P 31/10

(57) ABSTRACT

Provided herein are compositions comprising a C₁₋₄ alcohol, and a nonionic surfactant, and optionally including one or both of a preservative or an odorant, which are useful in solubilizing one or more active pharmaceutical ingredients. Also provided herein are aqueous compositions comprising an active pharmaceutical ingredient (e.g., one or more antifungals), a C₁₋₄ alcohol, and an excipient, and optionally including one or more of a preservative or a fragrance. Also provided herein are methods of solubilizing an antifungal active pharmaceutical ingredient in the compositions provided herein.

### CROSS REFERENCE TO RELATED APPLICATIONS

This application claims priority to U.S. Provisional Application No. 63/588,795 filed on Oct. 9, 2023, U.S. Provisional Application No. 63/599,949 filed on Nov. 16, 2023, and U.S. Provisional Application No. 63/670,307 filed on Jul. 12, 2024, each of which is incorporated herein by reference in its entirety.

### BACKGROUND

Active pharmaceutical ingredients (APIs), including antifungals, having negligible or imperceptible water solubility suffer from limited commercial use due in part to difficulties arising from issues relating to pharmacokinetics (e.g., poor bioavailability), manufacture, stability, formulation, and administration. There is a continuing need for aqueous solutions comprising APIs, including antifungals, that are not intrinsically water-soluble.

### SUMMARY

Provided herein are compositions comprising a solvent, and a nonionic surfactant, and optionally including one or both of a preservative or an odorant, which are useful in solubilizing one or more active pharmaceutical ingredients.

Also provided herein are aqueous compositions comprising an active pharmaceutical ingredient (e.g., one or more antifungals), a C₁₋₄ alcohol, and an excipient, and optionally including one or more of a preservative or a fragrance.

Also provided herein are methods of solubilizing an antifungal active pharmaceutical ingredient in the compositions provided herein.

Further provided are methods for treating and/or preventing antifungal infections. Also provided are kits comprising antifungal solutions described herein.

### DETAILED DESCRIPTION

Compositions provided herein are useful in solubilizing active pharmaceutical ingredients (APIs) that have little to no aqueous solubility. Many antifungals, such as nystatin, terbinafine, or fluconazole, are at best sparingly soluble in water. For example, tolnaftate is insoluble in water, only soluble at 1 part to 4000 in ethanol, 1 part to 55 in ether, 1 part to 9 in carbon tetrachloride, 1 part to 7 in acetone, and 1 part to 1.5 in chloroform. Antifungals like amphotericin B, clotrimazole, econazole, itraconazole, ketoconazole, miconazole, anidulafungine are also insoluble in water. Unfortunately, many solvents suitable for solubilizing APIs that are intrinsically not soluble in water are not suitable for administration to living subjects, e.g., due to solvent toxicity. It has been found that a mixture comprising an alcohol, at least one nonionic surfactant, and an API that is insoluble in water, or only sparingly soluble in water, can then be dispersed into water thereby rendering APIs—that are otherwise insoluble, or only sparingly soluble, in water-soluble in aqueous solution. Such aqueous API solutions are commercially useful, and needed, for continuing to provide effective treatment options for the various diseases or conditions for which the API is developed or approved.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.”

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound (i.e. API) described herein with another described component, which may be a carrier or a pharmaceutically acceptable carrier, respectively. The pharmaceutical composition facilitates administration of the at least one compound to a patient or subject. Multiple techniques of administering a composition exist including, but are not limited to, intravenous, oral, nasal, rectal, intravaginal, aerosol, parenteral, buccal, sublingual, ophthalmic, pulmonary, transdermal, and topical administration.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “monoterpene” refers to a class of terpenes having two isoprene units and the molecular formula C₁₀H₁₆, which may be linear (acyclic) or contain one or two rings (monocyclic and bicyclic).

The term “monoterpenoid” refers to a terpene containing an oxygen functionality or missing a methyl group otherwise present on the parent terpene.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

The term “% w/w” refers to percentage by weight of the referenced component based on the total weight of the composition.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. It is to be understood that the embodiments of this disclosure are illustrative. Each group member may be referred to and claimed individually or in any combination with other members of the group or other elements found herein. Furthermore, a recited member of a group may be included in, or excluded from, another recited group for reasons of convenience or patentability. Accordingly, the present disclosure is not limited to that precisely as shown and described.

References made to patent documents and printed publications throughout this specification serve as an incorporation by reference of their entire contents.

### Compositions

Provided herein are mixtures, comprising a C₁₋₄ alcohol and a nonionic surfactant, which are useful in preparing aqueous solutions of one or more active pharmaceutical ingredients (APIs) that are, independently, not soluble in water or are sparingly soluble in water (e.g., having a solubility of not more than about 1 mg/mL).

Provided herein are compositions comprising:
about 0.5 to about 2% w/w of an active pharmaceutical ingredient selected from one or more of amphotericin B, anidulafungine, clotrimazole, econazole, fluconazole, itraconazole, ketoconazole, miconazole, nystatin, terbinafine, or tolnaftate; and
a mixture comprising:
about 25% to about 60% w/w of a C₁₋₄ alcohol; and
about 19% to about 75% w/w of at least one nonionic surfactant.

In some embodiments, the C₁₋₄ alcohol is ethanol.

In some embodiments, the at least one nonionic surfactant is selected from a polyethylene glycol, polysorbate 20, or a combination thereof.

In some embodiments, the C₁₋₄ alcohol is ethanol; and the at least one nonionic surfactant is polyethylene glycol 300 or polysorbate 20, or a combination thereof. In some embodiments, the at least one nonionic surfactant is polyethylene glycol 300. In some embodiments, the at least one nonionic surfactant is polysorbate 20. In some embodiments, the at least one nonionic surfactant is a combination of polyethylene glycol 300 and polysorbate 20.

In some embodiments, the compositions comprise about 1% w/w of the active pharmaceutical ingredient.

In some embodiments, the mixture in the compositions comprises:
about 25% w/w of the C₁₋₄ alcohol;
about 74.5% w/w of the nonionic surfactant; and
about 0.5% w/w of a preservative.

In some embodiments, the mixture in the compositions comprises:
about 60% w/w of the C₁₋₄ alcohol;
about 39.8% w/w of the nonionic surfactant; and
about 0.2% w/w of a preservative.

In some embodiments, the mixture in the composition comprises:
about 37% w/w of the C₁₋₄ alcohol;
about 61% w/w of the at least one nonionic surfactant; and
about 0.5% w/w of a preservative.

In some embodiments, the mixture in the composition comprises:
about 25-60% w/w ethanol;
about 20-35% w/w polyethylene glycol 300;
about 19.8-39.5% w/w polysorbate 20; and
about 0.2-0.5% w/w methyl paraben.

In some embodiments, the mixture in the composition comprises:
A) about 60% w/w ethanol;
   about 20% w/w polyethylene glycol 300;
   about 19.8% w/w polysorbate 20; and
   about 0.2% w/w methyl paraben; or
B) about 25% w/w ethanol;
   about 35% w/w polyethylene glycol 300;
   about 39.5% w/w polysorbate 20; and
   about 0.5% w/w methyl paraben; or
C) about 60% w/w ethanol;
   about 39.5% w/w polysorbate 20; and
   about 0.5% w/w methyl paraben; or
D) about 60% w/w ethanol;
   about 35% w/w polyethylene glycol 300; and
   about 0.5% w/w methyl paraben; or
E) about 37% w/w ethanol;
   about 30% w/w polysorbate 20; and
   about 0.5% w/w methyl paraben;
F) about 37% w/w ethanol;
   about 31% w/w polyethylene glycol 300; and
   about 0.5% w/w methyl paraben; or
G) about 37% w/w ethanol;
   about 30% w/w polysorbate 20;
   about 31% w/w polyethylene glycol 300; and
   about 0.5% w/w methyl paraben.

In some embodiments, the compositions comprise a mixture comprising:
about 1% w/w of an API (e.g., an API suitable for topical therapeutic application) having a water solubility of less than 1 mg/mL;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of amphotericin B;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of anidulafungine;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of clotrimazole;
about 37% w/w ethanol;
about 31% w/w polyethylene glyccol 300;
about 30% w/w polysorbate 20; and about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of econazole;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of fluconazole;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of itraconazole;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of ketoconazole;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of miconazole;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of nystatin;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of terbinafine;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions comprise a mixture comprising: about 1% w/w of tolnaftate;
about 37% w/w ethanol;
about 31% w/w polyethylene glyccol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted about 1:1 to 1:150 in water.

In some embodiments, the compositions further comprise 0 to about 5% of an odorant. In some embodiments, the compositions further comprise 0 to about 1% of an odorant.

In some embodiments, the compositions are dispersed in a volume of water, thereby providing aqueous solutions of active pharmaceutical ingredients that are not soluble or sparingly soluble in water.

Also provided herein are compositions, comprising a C₁₋₄ alcohol, at least one nonionic surfactant, and an active pharmaceutical ingredient (API), wherein:
the composition is an aqueous composition;
the API is dissolved in the aqueous composition; and
the API has a water solubility of less than 1 mg/mL.

In some embodiments, provided herein are compositions comprising about 0.5% to about 2% w/w of an active pharmaceutical ingredient, water, and a mixture, the mixture comprising:
about 25% to about 60% w/w of the C₁₋₄ alcohol; and
about 19% to about 75% w/w of the at least one nonionic surfactant.

In some embodiments, provided herein are compositions comprising about 0.5% to about 2% w/w of an active pharmaceutical ingredient, water, and a mixture, the mixture comprising:
about 25% to about 60% w/w of the C₁₋₄ alcohol; and
about 20% to about 35% w/w of a polyethylene glycol;
about 19% to about 40% w/w of a polysorbate; and
about 0.2% to about 0.5% of a preservative.

In some embodiments, provided herein are compositions comprising about 1% w/w of an active pharmaceutical ingredient, water, and a mixture, the mixture comprising:
about 60% w/w of the C₁₋₄ alcohol; and
about 20% w/w of a polyethylene glycol;
about 19.8% w/w of a polysorbate; and
about 0.2% of a preservative.

In some embodiments, provided herein are compositions comprising about 1% w/w of an active pharmaceutical ingredient, water, and a mixture, the mixture comprising: about 25% w/w of the C₁₋₄ alcohol; and
about 35% w/w of a polyethylene glycol;
about 39.5% w/w of a polysorbate; and
about 0.5% of a preservative.

In some embodiments, provided herein are compositions comprising about 1% w/w of the active pharmaceutical ingredient, water, and a mixture, and optionally further comprising 0 to about 1% w/w of at least one odorant,
wherein the mixture comprises:
about 25% w/w of the C₁₋₄ alcohol;
about 74.5% w/w of the nonionic surfactant; and
about 0.5% w/w of a preservative.

In some embodiments, provided herein are compositions comprising about 1% w/w of the active pharmaceutical ingredient, water, and a mixture, and optionally further comprising 0 to about 1% w/w of at least one odorant,
wherein the mixture comprises:
about 60% w/w of the C₁₋₄ alcohol;
about 39.8% w/w of the nonionic surfactant; and
about 0.2% w/w of a preservative.

In some embodiments, provided herein are compositions comprising about 1% w/w of the active pharmaceutical ingredient, water, and a mixture, and optionally further comprising 0 to about 1% w/w of at least one odorant,
wherein the mixture comprises:
about 37% w/w of the C₁₋₄ alcohol;
about 61% w/w of the nonionic surfactant; and
about 0.5% w/w of a preservative.

In some embodiments, the API is pre-solubilized in a mixture comprising a C₁₋₄ alcohol and one or more nonionic surfactants, prior to inclusion of water, thereby allowing for the API having a water solubility of less than 1 mg/mL to remain solubilized in an aqueous composition. In some embodiments, the API is pre-solubilized in a mixture comprising a C₁-4 alcohol and one or more nonionic surfactants and forms a gel or paste or liquid which is then dispersed in water to form a liquid solution which is used for treatment as described in Example 51.

In some embodiments, the compositions described herein are liquid at about room temperature (about 20° C. to about 25° C.). In some embodiments, the compositions described herein are liquid at a range of atmospheric pressures, e.g., about—0.5 atm, or about 1 atm to about 3 atm pressures. In some embodiments, the compositions described herein do not include propellants such as butane, propane or isobutane. In some embodiments, the compositions described herein are substantially non-aqueous and are diluted in water at time of use. In some embodiments, “substantially non-aqueous” means the compositions comprise less than about 3% w/w, less than about 2% w/w, or less than about 1% w/w of water.

In some embodiments, the one or more API includes one or more of amphotericin B, anidulafungin, caspofungin, clotrimazole, econazole, fluconazole, itraconazole, ketoconazole, micafungin, miconazole, nystatin, terbinafine, or tolnaftate. In some embodiments, the one or more API includes one or more of amphotericin B, anidulafungin, clotrimazole, econazole, fluconazole, itraconazole, ketoconazole, miconazole, nystatin, terbinafine, or tolnaftate. In some embodiments, the one or more API includes tolnaftate.

In some embodiments, the one or more API includes amphotericin B. In some embodiments, the one or more API includes anidulafungin. In some embodiments, the one or more API includes clotrimazole. In some embodiments, the one or more API includes econazole. In some embodiments, the one or more API includes fluconazole. In some embodiments, the one or more API includes itraconazole. In some embodiments, the one or more API includes ketoconazole. In some embodiments, the one or more API includes miconazole. In some embodiments, the one or more API includes nystatin. In some embodiments, the one or more API includes terbinafine. In some embodiments, the one or more API includes nystatin. In some embodiments, the one or more API includes tolnaftate. In some embodiments, the one or more API includes undecylenic acid. In some embodiments, the one or more API includes hydrocortisone. In some embodiments, the API includes an antifungal (e.g., tolnaftate), undecylenic acid, hydrocortisone, or a combination thereof.

In some embodiments, the C₁₋₄ alcohol can be methanol, ethanol, propanol, isopropanol, butanol, an isobutanol, or a combination thereof. In some embodiments, the C₁₋₄ alcohol includes ethanol.

In some embodiments, the nonionic surfactant is a single nonionic surfactant, or a combination of nonionic surfactants. In some embodiments, the nonionic surfactants are selected from a polyether (e.g., a polyethylene glycol), a polysorbate (e.g., polysorbate 20), or a combination thereof. In some embodiments, the polyethylene glycol is a polyethylene glycol 300. In some embodiments, the polyethylene glycol can have a PEG terminus of a primary hydroxyl (—OH), end-capped with a C₁₋₄ alkoxy (e.g., methoxy (—OCH₃)), or a combination thereof. In some embodiments, the polysorbate includes polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), sorbitan monolaurate, sorbitan monostearate, sorbitan tristearate, or sorbitan monooleate, or a combination thereof. In some embodiments, nonionic surfactants may be polyglycerol alkyl ethers, glucosyl dialkyl ethers, crownethers, ester-linked surfactants, polyoxyethylene alkyl ethers, or ether of natural fatty alcohols.

In some embodiments, the nonionic surfactant includes polyethylene glycol 300. In some embodiments, the nonionic surfactant includes polysorbate 20. In some embodiments, the nonionic surfactant includes polyethylene glycol 300 and polysorbate 20.

In some embodiments of the compositions,
the C₁₋₄ alcohol is ethanol;
the nonionic surfactant is selected from polyethylene glycol 300 or polysorbate 20, or a combination thereof; and
the active pharmaceutical ingredient is selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin.

In some embodiments, the compositions further comprise a preservative. In some embodiments, the mixture of a C₁-4 alcohol and one or more nonionic surfactants included in the compositions further comprises a preservative. In some embodiments, mixture comprises about 0.2% to about 0.5% w/w of a preservative. In some of such embodiments, the preservative includes a C₁₋₄ alkyl ester of para-hydroxybenzoic acid. In some embodiments, the preservative includes methyl 4-hydroxybenzoate (methylparaben). In some embodiments, the preservative includes one or more of methylparaben, ethylparaben, propylparaben, butylparaben, heptylparaben, isobutylparaben, isopropylparaben, benzylparaben, or a salt thereof (e.g., a sodium salt thereof).

In some embodiments, the compositions herein may include one or more odorants. The one or more odorants may provide a fragrance to the compositions, which fragrance is identifiable to an individual sensing via olfaction the one or more odorants. In some embodiments, the one or more odorants can include at least one monoterpene or monoterpenoid. In some embodiments, the one or more odorants include, and thereby the compositions may include, one or more of menthol, limonene, carvone, hinokitiol, or linalool.

In some embodiments, the compositions comprise about 1% w/w of the active pharmaceutical ingredient, water, and a mixture, and optionally further comprise 0 to about 1% w/w of at least one odorant,
wherein the mixture comprises:
about 25% to about 60% w/w of the C₁₋₄ alcohol;
about 20% to about 40% w/w of the nonionic surfactant; and
about 0.2% to about 0.5% w/w of a preservative.

In some embodiments, the compositions comprise about 1% w/w of the active pharmaceutical ingredient, water, and a mixture, and optionally further comprise 0 to about 1% w/w of at least one odorant,
wherein the mixture comprises:
about 25% w/w of the C₁₋₄ alcohol;
about 74.5% w/w of the nonionic surfactant; and
about 0.5% w/w of a preservative.

In some embodiments, the compositions comprise about 1% w/w of the active pharmaceutical ingredient, water, and a mixture, and optionally further comprise 0 to about 1% w/w of at least one odorant,
wherein the mixture comprises:
about 60% w/w of the C₁₋₄ alcohol;
about 39.8% w/w of the nonionic surfactant; and
about 0.2% w/w of a preservative.

In some embodiments of the compositions herein, the compositions further comprise undecylenic acid. In some of such embodiments, the compositions comprise from about 0.001% w/w to about 25% w/w of undecylenic acid. In some of such embodiments, the compositions comprise from about 0.001% w/w to about 5% w/w, 0.001% w/w to about 10% w/w, 0.001% w/w to about 15% w/w, or 0.001% w/w to about 20% w/w of undecylenic acid. In some of such embodiments, the compositions further comprise from about 0.01% w/w to about 5% w/w, 0.01% to about 3% w/w, or 0.01% to about 1% w/w of undecylenic acid. In some of such embodiments, the compositions further comprise from about 0.1% w/w to about 5% w/w, 0.1% to about 3% w/w, or 0.1% to about 1% w/w of undecylenic acid.

In some embodiments of the compositions herein, the compositions further comprise hydrocortisone. In some of such embodiments, the compositions comprise from about 0.001% w/w to about 25% w/w of hydrocortisone. In some of such embodiments, the compositions comprise from about 0.001% w/w to about 5% w/w, 0.001% w/w to about 10% w/w, 0.001% w/w to about 15% w/w, or 0.001% w/w to about 20% w/w of hydrocortisone. In some of such embodiments, the compositions further comprise from about 0.01% w/w to about 5% w/w, 0.01% to about 3% w/w, or 0.01% to about 1% w/w of hydrocortisone. In some of such embodiments, the compositions further comprise from about 0.1% w/w to about 5% w/w, 0.1% to about 3% w/w, or 0.1% to about 1% w/w of hydrocortisone.

In some embodiments of the compositions herein, the nonionic surfactant comprises about 20% to about 35% w/w of polyethylene glycol (e.g., PEG 300). In some embodiments of the compositions herein, the nonionic surfactant comprises about 19.8% to about 39.5% of a polysorbate (e.g., polysorbate 20). In some embodiments of the compositions herein, the nonionic surfactant comprises about 20% to about 35% w/w of polyethylene glycol (e.g., PEG 300) and about 19.8% to about 39.5% of a polysorbate (e.g., polysorbate 20). In some embodiments of the compositions herein, the compositions comprise about 0.1 to 20% w/w API (e.g., amphotericin B, anidulafungin, clotrimazole, econazole, fluconazole, itraconazole, ketoconazole, miconazole, nystatin, terbinafine, or tolnaftate). In some embodiments of the compositions herein, the compositions comprise about 0.5, 1, 2, 3, 4, 5, 6, 7, 8, 9, 10, 11, 12, 13, 14, 15, 16, 17, 18, 19, or 20% w/w amphotericin B, anidulafungin, clotrimazole, econazole, fluconazole, itraconazole, ketoconazole, miconazole, nystatin, terbinafine, or tolnaftate. In some embodiments, the one or more API is dissolved in the composition. In some embodiments, the compositions are clear. In some embodiments, the compositions are colorless. In some embodiments, the compositions have been filtered through a 0.45 μm filter.

In some embodiments, the compositions herein are antifungal compositions. In some embodiments, the compositions herein are topical compositions.

In some embodiments, the compositions herein are solutions. In some embodiments, the compositions herein are liquid solutions.

In some embodiments, the compositions provided herein comprise:
ethanol;
a nonionic surfactant selected from polyethylene glycol 300 or polysorbate 20, or a combination thereof;
menthol; and
methyl 4-hydroxybenzoate;
wherein the API is selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin.

In some embodiments, the compositions provided herein comprise:
water;
ethanol;
a nonionic surfactant selected from polyethylene glycol 300 or polysorbate 20, or a combination thereof;
menthol; and
methyl 4-hydroxybenzoate;
wherein the API is selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin.

In some embodiments, the compositions provided herein comprise:
ethanol;
polyethylene glycol 300;
menthol;
methyl 4-hydroxybenzoate;
amphotericin B, wherein the amphotericin B is dissolved or the composition is clear.

In some embodiments, the compositions provided herein comprise:
water;
ethanol;
polyethylene glycol 300;
menthol; methyl 4-hydroxybenzoate;
amphotericin B, wherein the amphotericin B is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w amphotericin B, wherein the amphotericin B is dissolved or the composition is clear.

In some embodiments, the compositions provided herein comprise:
water;
ethanol;
polyethylene glycol 300;
menthol;
methyl 4-hydroxybenzoate;
about 0.5% w/w amphotericin B, wherein the amphotericin B is dissolved or the composition is clear.

In some embodiments, the compositions provided herein comprise:
water;
ethanol;
polyethylene glycol 300;
menthol;
methyl 4-hydroxybenzoate;
about 1.0 w/w amphotericin B, wherein the amphotericin B is dissolved or the composition is clear.

In some embodiments, the compositions provided herein comprise:
water;
ethanol;
polyethylene glycol 300;
menthol;
methyl 4-hydroxybenzoate;
about 1.5 w/w amphotericin B, wherein the amphotericin B is dissolved or the composition is clear.

In some embodiments, the compositions provided herein comprise:
water;
ethanol;
polyethylene glycol 300;
menthol;
methyl 4-hydroxybenzoate;
about 2.0 w/w amphotericin B, wherein the amphotericin B is dissolved or the composition is clear.

In some embodiments, the compositions provided herein comprise:
ethanol;
polyethylene glycol 300;
menthol;
methyl 4-hydroxybenzoate;
anidulafungine, wherein the anidulafungine is dissolved or the composition is clear.

In some embodiments, the compositions provided herein comprise:
water;
ethanol;
polyethylene glycol 300;
menthol;
methyl 4-hydroxybenzoate;
anidulafungine, wherein the anidulafungine is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w anidulafungine, wherein the anidulafungine is dissolved or the composition is clear.

In some embodiments, the compositions provided herein comprise:
water;
ethanol;
polyethylene glycol 300;
menthol;
methyl 4-hydroxybenzoate;
about 0.5% w/w anidulafungine, wherein the anidulafungine is dissolved or the composition is clear.

In some embodiments, the compositions provided herein comprise:
water;
ethanol;
polyethylene glycol 300;
menthol;
methyl 4-hydroxybenzoate;
about 1.0 w/w anidulafungine, wherein the anidulafungine is dissolved or the composition is clear.

In some embodiments, the compositions provided herein comprise:
water;
ethanol;
polyethylene glycol 300;
menthol;
methyl 4-hydroxybenzoate;
about 1.5 w/w anidulafungine, wherein the anidulafungine is dissolved or the composition is clear.

In some embodiments, the compositions provided herein comprise:
water;
ethanol;
polyethylene glycol 300;
menthol;
methyl 4-hydroxybenzoate;
about 2.0 w/w anidulafungine, wherein the anidulafungine is dissolved or the composition is clear.

In some embodiments, the compositions provided herein comprise:
ethanol;
polyethylene glycol 300;
menthol;
methyl 4-hydroxybenzoate;
clotrimazole, wherein the clotrimazole is dissolved or the composition is clear.

In some embodiments, the compositions provided herein comprise:
water;
ethanol;
polyethylene glycol 300;
menthol;
methyl 4-hydroxybenzoate;
clotrimazole, wherein the clotrimazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w clotrimazole, wherein the clotrimazole is dissolved or the composition is clear.

In some embodiments, the compositions provided herein comprise:
water;
ethanol;
polyethylene glycol 300;
menthol;
methyl 4-hydroxybenzoate;
about 0.5% w/w clotrimazole, wherein the clotrimazole is dissolved or the composition is clear.

In some embodiments, the compositions provided herein comprise:
water;
ethanol;
polyethylene glycol 300;
menthol;
methyl 4-hydroxybenzoate;
about 1.0 w/w clotrimazole, wherein the clotrimazole is dissolved or the composition is clear.

In some embodiments, the compositions provided herein comprise:
water;
ethanol;
polyethylene glycol 300;
menthol;
methyl 4-hydroxybenzoate;
about 1.5 w/w clotrimazole, wherein the clotrimazole is dissolved or the composition is clear.

In some embodiments, the compositions provided herein comprise:
water;
ethanol;
polyethylene glycol 300;
menthol;
methyl 4-hydroxybenzoate;
about 2.0 w/w clotrimazole, wherein the clotrimazole is dissolved or the composition is clear.

In some embodiments, the compositions provided herein comprise:
ethanol;
polyethylene glycol 300;
menthol;
methyl 4-hydroxybenzoate;
econazole, wherein the econazole is dissolved or the composition is clear.

In some embodiments, the compositions provided herein comprise:
water;
ethanol;
polyethylene glycol 300;
menthol;
methyl 4-hydroxybenzoate;
econazole, wherein the econazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w econazole, wherein the econazole is dissolved or the composition is clear.

In some embodiments, the compositions provided herein comprise:
water;
ethanol;
polyethylene glycol 300;
menthol;
methyl 4-hydroxybenzoate;
about 0.5% w/w econazole, wherein the econazole is dissolved or the composition is clear.

In some embodiments, the compositions provided herein comprise:
water;
ethanol;
polyethylene glycol 300;
menthol;
methyl 4-hydroxybenzoate;
about 1.0 w/w econazole, wherein the econazole is dissolved or the composition is clear.

In some embodiments, the compositions provided herein comprise:
water;
ethanol;
polyethylene glycol 300;
menthol;
methyl 4-hydroxybenzoate;
about 1.5 w/w econazole, wherein the econazole is dissolved or the composition is clear.

In some embodiments, the compositions provided herein comprise:
water;
ethanol;
polyethylene glycol 300;
menthol;
methyl 4-hydroxybenzoate;
about 2.0 w/w econazole, wherein the econazole is dissolved or the composition is clear.

In some embodiments, the compositions provided herein comprise:
ethanol;
polyethylene glycol 300;
menthol;
methyl 4-hydroxybenzoate;
fluconazole, wherein the fluconazole is dissolved or the composition is clear.

In some embodiments, the compositions provided herein comprise:
water;
ethanol;
polyethylene glycol 300;
menthol;
methyl 4-hydroxybenzoate;
fluconazole, wherein the fluconazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w fluconazole, wherein the fluconazole is dissolved or the composition is clear.

In some embodiments, the compositions provided herein comprise:
water;
ethanol;
polyethylene glycol 300;
menthol;
methyl 4-hydroxybenzoate;
about 0.5% w/w fluconazole, wherein the fluconazole is dissolved or the composition is clear.

In some embodiments, the compositions provided herein comprise:
water;
ethanol;
polyethylene glycol 300;
menthol;
methyl 4-hydroxybenzoate;
about 1.0 w/w fluconazole, wherein the fluconazole is dissolved or the composition is clear.

In some embodiments, the compositions provided herein comprise:
water;
ethanol;
polyethylene glycol 300;
menthol;
methyl 4-hydroxybenzoate;
about 1.5 w/w fluconazole, wherein the fluconazole is dissolved or the composition is clear.

In some embodiments, the compositions provided herein comprise:
water;
ethanol;
polyethylene glycol 300;
menthol;
methyl 4-hydroxybenzoate;
about 2.0 w/w fluconazole, wherein the fluconazole is dissolved or the composition is clear.

In some embodiments, the compositions provided herein comprise:
ethanol;
polyethylene glycol 300;
menthol;
methyl 4-hydroxybenzoate;
itraconazole, wherein the itraconazole is dissolved or the composition is clear.

In some embodiments, the compositions provided herein comprise:
water;
ethanol;
polyethylene glycol 300;
menthol;
methyl 4-hydroxybenzoate;
itraconazole, wherein the itraconazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w itraconazole, wherein the itraconazole is dissolved or the composition is clear.

In some embodiments, the compositions provided herein comprise:
water;
ethanol;
polyethylene glycol 300;
menthol;
methyl 4-hydroxybenzoate;
about 0.5% w/w itraconazole, wherein the itraconazole is dissolved or the composition is clear.

In some embodiments, the compositions provided herein comprise:
water;
ethanol;
polyethylene glycol 300;
menthol;
methyl 4-hydroxybenzoate;
about 1.0 w/w itraconazole, wherein the itraconazole is dissolved or the composition is clear.

In some embodiments, the compositions provided herein comprise:
water;
ethanol;
polyethylene glycol 300;
menthol;
methyl 4-hydroxybenzoate;
about 1.5 w/w itraconazole, wherein the itraconazole is dissolved or the composition is clear.

In some embodiments, the compositions provided herein comprise:
water;
ethanol;
polyethylene glycol 300;
menthol;
methyl 4-hydroxybenzoate;
about 2.0 w/w itraconazole, wherein the itraconazole is dissolved or the composition is clear.

In some embodiments, the compositions provided herein comprise:
ethanol;
polyethylene glycol 300;
menthol;
methyl 4-hydroxybenzoate;
ketoconazole, wherein the ketoconazole is dissolved or the composition is clear.

In some embodiments, the compositions provided herein comprise:
water;
ethanol;
polyethylene glycol 300;
menthol;
methyl 4-hydroxybenzoate;
ketoconazole, wherein the ketoconazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w ketoconazole, wherein the ketoconazole is dissolved or the composition is clear.

In some embodiments, the compositions provided herein comprise:
water;
ethanol;
polyethylene glycol 300;
menthol;
methyl 4-hydroxybenzoate;
about 0.5% w/w ketoconazole, wherein the ketoconazole is dissolved or the composition is clear.

In some embodiments, the compositions provided herein comprise:
water;
ethanol;
polyethylene glycol 300;
menthol;
methyl 4-hydroxybenzoate;
about 1.0 w/w ketoconazole, wherein the ketoconazole is dissolved or the composition is clear.

In some embodiments, the compositions provided herein comprise:
water;
ethanol;
polyethylene glycol 300;
menthol;
methyl 4-hydroxybenzoate;
about 1.5 w/w ketoconazole, wherein the ketoconazole is dissolved or the composition is clear.

In some embodiments, the compositions provided herein comprise:
water;
ethanol;
polyethylene glycol 300;
menthol;
methyl 4-hydroxybenzoate;
about 2.0 w/w ketoconazole, wherein the ketoconazole is dissolved or the composition is clear.

In some embodiments, the compositions provided herein comprise:
ethanol;
polyethylene glycol 300;
menthol;
methyl 4-hydroxybenzoate;
miconazole, wherein the miconazole is dissolved or the composition is clear.

In some embodiments, the compositions provided herein comprise:
water;
ethanol;
polyethylene glycol 300;
menthol;
methyl 4-hydroxybenzoate;
miconazole, wherein the miconazole is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w miconazole, wherein the miconazole is dissolved or the composition is clear.

In some embodiments, the compositions provided herein comprise:
water;
ethanol;
polyethylene glycol 300;
menthol;
methyl 4-hydroxybenzoate;
about 0.5% w/w miconazole, wherein the miconazole is dissolved or the composition is clear.

In some embodiments, the compositions provided herein comprise:
water;
ethanol;
polyethylene glycol 300;
menthol;
methyl 4-hydroxybenzoate;
about 1.0 w/w miconazole, wherein the miconazole is dissolved or the composition is clear.

In some embodiments, the compositions provided herein comprise:
water;
ethanol;
polyethylene glycol 300;
menthol;
methyl 4-hydroxybenzoate;
about 1.5 w/w miconazole, wherein the miconazole is dissolved or the composition is clear.

In some embodiments, the compositions provided herein comprise:
water;
ethanol;
polyethylene glycol 300;
menthol;
methyl 4-hydroxybenzoate;
about 2.0 w/w miconazole, wherein the miconazole is dissolved or the composition is clear.

In some embodiments, the compositions provided herein comprise:
ethanol;
polyethylene glycol 300;
menthol;
methyl 4-hydroxybenzoate;
nystatin, wherein the nystatin is dissolved or the composition is clear.

In some embodiments, the compositions provided herein comprise:
water;
ethanol;
polyethylene glycol 300;
menthol;
methyl 4-hydroxybenzoate;
nystatin, wherein the nystatin is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w nystatin, wherein the nystatin is dissolved or the composition is clear.

In some embodiments, the compositions provided herein comprise:
water;
ethanol;
polyethylene glycol 300;
menthol;
methyl 4-hydroxybenzoate;
about 0.5% w/w nystatin, wherein the nystatin is dissolved or the composition is clear.

In some embodiments, the compositions provided herein comprise:
water;
ethanol;
polyethylene glycol 300;
menthol;
methyl 4-hydroxybenzoate;
about 1.0 w/w nystatin, wherein the nystatin is dissolved or the composition is clear.

In some embodiments, the compositions provided herein comprise:
water;
ethanol;
polyethylene glycol 300;
menthol;
methyl 4-hydroxybenzoate;
about 1.5 w/w nystatin, wherein the nystatin is dissolved or the composition is clear.

In some embodiments, the compositions provided herein comprise:
water;
ethanol;
polyethylene glycol 300;
menthol;
methyl 4-hydroxybenzoate;
about 2.0 w/w nystatin, wherein the nystatin is dissolved or the composition is clear.

In some embodiments, the compositions provided herein comprise:
ethanol;
polyethylene glycol 300;
menthol;
methyl 4-hydroxybenzoate;
terbinafine, wherein the terbinafine is dissolved or the composition is clear.

In some embodiments, the compositions provided herein comprise:
water;
ethanol;
polyethylene glycol 300;
menthol;
methyl 4-hydroxybenzoate;
terbinafine, wherein the terbinafine is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w terbinafine, wherein the terbinafine is dissolved or the composition is clear.

In some embodiments, the compositions provided herein comprise:
water;
ethanol;
polyethylene glycol 300;
menthol;
methyl 4-hydroxybenzoate;
about 0.5% w/w terbinafine, wherein the terbinafine is dissolved or the composition is clear.

In some embodiments, the compositions provided herein comprise:
water;
ethanol;
polyethylene glycol 300;
menthol;
methyl 4-hydroxybenzoate;
about 1.0% w/w terbinafine, wherein the terbinafine is dissolved or the composition is clear.

In some embodiments, the compositions provided herein comprise:
water;
ethanol;
polyethylene glycol 300;
menthol;
methyl 4-hydroxybenzoate;
about 1.5% w/w terbinafine, wherein the terbinafine is dissolved or the composition is clear.

In some embodiments, the compositions provided herein comprise:
water;
ethanol;
polyethylene glycol 300;
menthol;
methyl 4-hydroxybenzoate;
about 2.0% w/w terbinafine, wherein the terbinafine is dissolved or the composition is clear.

In some embodiments, the compositions provided herein comprise:
ethanol;
polyethylene glycol 300;
menthol;
methyl 4-hydroxybenzoate;
tolnaftate, wherein the tolnaftate is dissolved or the composition is clear.

In some embodiments, the compositions provided herein comprise:
water;
ethanol;
polyethylene glycol 300;
menthol;
methyl 4-hydroxybenzoate;
tolnaftate, wherein the tolnaftate is dissolved or the composition is clear.

In some embodiments, the compositions provided herein comprise:
ethanol;
polyethylene glycol 300;
menthol;
methyl 4-hydroxybenzoate;
about 0.5% w/w to about 20% w/w tolnaftate, wherein the tolnaftate is dissolved or the composition is clear.

In some embodiments, the compositions provided herein comprise:
water;
ethanol;
polyethylene glycol 300;
menthol;
methyl 4-hydroxybenzoate;
about 0.5% w/w tolnaftate, wherein the tolnaftate is dissolved or the composition is clear.

In some embodiments, the compositions provided herein comprise:
water;
ethanol;
polyethylene glycol 300;
menthol;
methyl 4-hydroxybenzoate;
about 1.0 w/w tolnaftate, wherein the tolnaftate is dissolved or the composition is clear.

In some embodiments, the compositions provided herein comprise:
water;
ethanol;
polyethylene glycol 300;
menthol;
methyl 4-hydroxybenzoate;
about 1.5 w/w tolnaftate, wherein the tolnaftate is dissolved or the composition is clear.

In some embodiments, the compositions provided herein comprise:
water;
ethanol;
polyethylene glycol 300;
menthol;
methyl 4-hydroxybenzoate;
about 2.0 w/w tolnaftate, wherein the tolnaftate is dissolved or the composition is clear.

In some embodiments, the C₁₋₄ alcohol is a specially denatured (SD) C₁₋₄ alcohol. In some embodiments the ethanol referred to herein is provided as SD alcohol 40 or SD alcohol 30.

In some embodiments, the compositions comprise about 10% to 70% w/w C₁₋₄ alcohol (e.g., ethanol, e.g., SD alcohol 30 or 40), about 20% to 75% w/w of one or more nonionic surfactants, about 0.1% to 1% w/w menthol, about 0.1% to 0.5% w/w methyl 4-hydroxybenzoate, and about 0.5% to 2% w/w API.

In some embodiments, the compositions comprise about 25% to 60% w/w C₁₋₄ alcohol (e.g., ethanol, e.g., SD alcohol 30 or 40), about 20% to 35% w/w polyethylene glycol 300, about 0.1% to 1% w/w menthol, about 0.1% to 0.5% w/w methyl 4-hydroxybenzoate, and about 0.5% to 2% w/w API including amphotericin B, anidulafungine, clotrimazole, econazole, fluconazole, itraconazole, ketoconazole, miconazole, nystatin, terbinafine, or tolnaftate, optionally further including one or more of caspofungin or micafungin.

In some embodiments, the compositions comprise about 25% to 60% w/w C₁₋₄ alcohol (e.g., ethanol, e.g., SD alcohol 30 or 40), about 20% to 40% w/w polysorbate 20, about 0.1% to 1% w/w menthol, about 0.1% to 0.5% w/w methyl 4-hydroxybenzoate, and about 0.5% to 2% w/w API including amphotericin B, anidulafungine, clotrimazole, econazole, fluconazole, itraconazole, ketoconazole, miconazole, nystatin, terbinafine, or tolnaftate, optionally further including one or more of caspofungin or micafungin.

In some embodiments, the compositions herein have a pH of about 5.5-7.0 at about 25° C. In some embodiments, the compositions herein have a pH of about 5.5-6.5 at about 25° C. In some embodiments, the compositions herein have a pH of about 6.0-6.5 at about 25° C. In some embodiments, the compositions herein have a pH of about 6 at about 25° C. In some embodiments, the compositions herein have a pH of about 6.2 at about 25° C.

In some embodiments, the C₁₋₄ alcohol (e.g., ethanol (e.g., SD alcohol 30 or SD alcohol 40)) is present in the compositions at about 8% w/w, about 10% w/w, about 12% w/w, about 14% w/w, about 16% w/w, about 18% w/w, about 20% w/w, about 22% w/w, about 24% w/w, about 26% w/w, about 28% w/w, about 30% w/w, about 32% w/w, about 34% w/w, about 36% w/w, about 38% w/w, about 40% w/w, about 42% w/w, about 44% w/w, about 46% w/w, about 48% w/w, about 50% w/w, about 52% w/w, about 54% w/w, about 56% w/w, about 58% w/w, about 60% w/w, about 62% w/w, about 64% w/w, about 66% w/w, about 68% w/w, about 70% w/w, about 72% w/w, about 74% w/w, about 76% w/w, about 78% w/w, or about 80% w/w.

In some embodiments, the nonionic surfactant (e.g., polyether (e.g., polyethylene glycol 300) or polysorbate (e.g., polysorbate 20)) or a combination thereof, is present in the compositions at about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 22% w/w, about 25% w/w, about 27% w/w, about 30% w/w, about 32% w/w, about 35% w/w, about 37% w/w, about 40% w/w, about 42% w/w, about 45% w/w, about 47% w/w, about 50% w/w, about 52% w/w, about 55% w/w, about 57% w/w, about 60% w/w, about 62% w/w, about 65% w/w, about 67% w/w, about 70% w/w, about 72% w/w, about 75% w/w, about 77% w/w, or about 80% w/w.

In some embodiments, the odorant (e.g., monoterpene or monoterpenoid (e.g., menthol)) is present in the compositions at about 0.05% w/w, about 0.06% w/w, about 0.07% w/w, about 0.08% w/w, about 0.09% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, or about 1.5% w/w, about 1.6% w/w, about 1.7% w/w, about 1.8% w/w, about 1.9% w/w, or about 2.0% w/w.

In some embodiments, the preservative (e.g., the C₁₋₄ alkyl ester of para-hydroxybenzoic acid (e.g., methyl 4-hydroxybenzoate)) is present in the compositions at about 0.05% w/w, about 0.06% w/w, about 0.07% w/w, about 0.08% w/w, about 0.09% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, or about 1.5% w/w.

In some embodiments, the API (e.g., amphotericin B, anidulafungin, caspofungin, clotrimazole, econazole, fluconazole, itraconazole, ketoconazole, micafungin, miconazole, nystatin, terbinafine, or tolnaftate) is present in the compositions at about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, or about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, or about 3.0% w/w.

In some embodiments, the compositions herein do not include pulegone, myrcene, or both.

In some embodiments, the non-aqueous mixture or composition herein comprising dissolved API, C₁₋₄ alcohol, and nonionic surfactant(s) are diluted about 1:1, 1:10 (about 1 part non-aqueous mixture to about 10 parts water), 1:50, 1:100, 1:130, or 1:150 in water to form a solution useful for soaking a portion of a subject's skin in need thereof. In some embodiments, about 1 ounce of the non-aqueous mixture or composition herein comprising dissolved API, C₁₋₄ alcohol, and nonionic surfactant(s) is diluted in about 1 gallon of water and mixed to form a homogeneous aqueous solution comprising the API.

In some embodiments, the compositions are provided in a patch that is configured to release the composition onto a surface (e.g., skin) that the patch is contacting. A dermal patch or skin patch comprises a medicated adhesive patch placed on the skin. The patch may comprise an adhesive strip with backing that can be peeled off. The composition comprising an active agent is adsorbed onto a pad which is affixed to the adhesive strip underneath the backing. The pad may be a filter paper, a gauze, or any other suitable material capable of absorbing and retaining the composition until the backing is removed and the dermal patch is affixed to the affected area on the skin. The medication in the composition adsorbed onto the patch may thereby remain in contact with the affected area on the skin and/or be subsequently absorbed into the surface of the skin, providing local non-systemic delivery to the affected area.

In some embodiments, the compositions described herein are diluted in water. In such embodiments, the antifungal soaks described herein can prophylactically reduce or prevent occurrence of a fungal infection (e.g., in the nails) in a subject undergoing manicures or pedicures.

### Methods

In some embodiments, the compositions herein are prepared by a method, comprising combining the components without water (e.g., by using non-aqueous solvents and/or surfactants) to prepare a mixture thereof, followed by combining the mixture with water such that the API (e.g., amphotericin B, anidulafungin, clotrimazole, econazole, fluconazole, itraconazole, ketoconazole, miconazole, nystatin, terbinafine, or tolnaftate, optionally further including caspofungin or micafungin) is dissolved therein and forms the aqueous composition. The aqueous composition may optionally be filtered, e.g., through a 0.45 μm filter. In some embodiments, the methods result in a clear or colorless, or clear and colorless, solution.

Also provided herein are methods of administration of the compositions herein to a subject in need thereof.

In some embodiments, provided herein are methods of treating a disease or condition in a subject in need thereof, comprising administration of a composition herein to the subject, wherein the disease or condition is one that the API is approved for treating.

In some embodiments, provided herein are methods of treating a fungal infection in a subject in need thereof, comprising administration of a composition provided herein to the subject. In some embodiments, the fungal infection is a skin infection. In some embodiments, the fungal infection is a nail infection. Also provided herein are methods for preventing fungal infection in a subject in need thereof (e.g., a subject who undergoes manicures or pedicures and is likely to contract a fungal infection).

In some embodiments, provided herein are methods of delaying the onset of a fungal infection in a subject in need thereof, comprising administration of a composition provided herein to the subject.

In some embodiments, provided herein are prophylactic methods, comprising administration of a composition provided herein to a subject in need thereof. In some embodiments the subject suffers from a fungal infection. In some embodiments, the administration is topical administration to a surface on the subject's skin or nail that includes the topical fungal infection.

In some embodiments, provided herein are methods of treating a fungal condition or infection (e.g., dermatophytosis or mycosis or onychomychosis) in a subject in need thereof, comprising administration of the composition described herein to the subject. In some embodiments, the fungal condition or infection is a topical fungal condition or infection. In some embodiments the fungal condition or infection is subcutaneous or systemic.

In some embodiments of the methods herein, the administration is topical administration. In some embodiments, the administration is to a surface of skin of the subject. In some embodiments, the administration is to a surface of a nail of the subject. In some embodiments, the surface to which the compositions may be applied include nails, palms of hands, scalp, soles of feet, ankles, wrists, ears, genital areas, pubic region, arm pits, back of knees, fat folds or any other area prone to developing a topical fungal infection.

In some embodiments, provided herein are methods of treating athlete's foot (dermatophytosis) in a subject in need thereof, comprising administration of a composition provided herein to the subject. In some embodiments, provided herein are methods of reducing or alleviating itching, burning, scaling, irritation, soreness, or a combination thereof, in a subject in need thereof, comprising administration of a composition provided herein to the subject. In some embodiments, “administration” refers to topical administration, e.g., a subject may contact the affected area with an aqueous solution described herein.

In some embodiments, provided herein are methods of reducing or alleviating itching, burning, scaling, irritation, soreness, or a combination thereof, associated with athletes foot, in a subject in need thereof, comprising contacting a composition provided herein with the affected area (on the skin) in a subject in need thereof.

In some embodiments, provided herein are methods of treating jock itch in a subject in need thereof, comprising administration of a composition provided herein to the subject.

In some embodiments, provided herein are methods of treating ringworm in a subject in need thereof, comprising administration of a composition provided herein to the subject.

In some embodiments, provided herein are methods of treating a yeast infection in a subject in need thereof, comprising administration of a composition provided herein to the subject.

In some embodiments of the methods herein, administration of an API-containing composition herein treats, or prophylactically addresses, one or more of the following: aspergillosis; blastomycosis; Candida auris infection; Candida infections of the mouth, throat, or esophagus (e.g., thrush); candidiasis (e.g., topical candidiasis, invasive, or vaginal candidiasis); coccidioidomycosis (Valley Fever); Cryptococcus gattii infection; Cryptococcus neoformans infection; fungal eye infections; fungal nail infections; healthcare-associated fungal meningitis; histoplasmosis; mucormycosis; mycetoma; paracoccidioidomycosis; pneumocystis pneumonia (PCP); ringworm; sporotrichosis; or talaromycosis. In some embodiments of the methods herein, administration of an API-containing composition herein treats, or prophylactically addresses, one or more of the following infections: Epidermophyton, Malassezia, Microsporum, or Trichophyton. In some embodiments of the methods herein, administration of an API-containing composition herein treats, or prophylactically addresses, one or more of the following: dandruff, seborrhoeic dermatitis, folliculitis, papillomatosis, or tinea (pityriasis) versicolor.

In some embodiments, the subject suffers from a weakened immune system and the methods described herein treat any fungal infection associated with a weakened immune system. In some embodiments, the weakened immune system is a result of a condition such as HIV, cancer, organ transplant, or certain medications associated with weakening the immune system or treating a disease that causes a weakened immune system. In some embodiments, the subject suffers from a fungal infection that is comorbid with diabetes, pneumonia, influenza, covid infection, bronchitis, hepatitis, tuberculosis, any other viral or bacterial infection, hyperhidrosis, peripheral vascular disease, psoriasis, an injury to the nail, or the fungal infection is a nosocomial infection. In some embodiments, the fungal infection is a yeast infection. In some embodiments, the fungal infection is onychomycosis including infections caused by dermatophytes (tinea unguium), yeasts, or saprophytic mold.

In some embodiments, provided is a method comprising (1) contacting an area of fungal infection (e.g., hands, feet) with a soaking solution prepared by diluting, in water, a non-aqueous composition (e.g., according to Example 57) comprising: about 1% w/w of tolnaftate, about 36.7% w/w of ethanol, about 30.9% w/w of polyethylene glycol, about 0.5% w/w of methylparaben, about 30% w/w of polysorbate, fragrance, and solvent to qs 100%; and (2) applying to the area of fungal infection (e.g., nails on hands or feet) a composition comprising: about 0.1% to about 1% w/w of undecylenic acid, about 90.5% w/w 2 ethyl cyanoacrylate, about 9% w/w polymethylmethacrylate, and about 0.4% w/w hydroquinone.

### Dosages and Regimens

Effective doses or amounts of the API or compositions may vary, depending on the conditions treated, the severity of the condition, the sex, age and general health condition of the subject, excipient usage, and the possibility of co-usage with other therapeutic treatments such as use of other agents and the judgment of the treating physician.

In some embodiments, an effective amount of the API or compositions, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of the API or compositions is about 0.1 mg/kg, about 0.5 mg/kg, about 1 mg/kg, about 2 mg/kg, or about 5 mg/kg.

In some embodiments, the compositions are administered on a daily basis (e.g., as a single dose or as two or more divided doses, e.g., once daily, twice daily, thrice daily, etc.) or on a non-daily basis (e.g., every other day, every two days, every three days, once weekly, twice weekly, once every two weeks, once a month, etc.). In some embodiments, the administration occurs on a daily or weekly basis for two or more weeks.

In some embodiments, each administration is, independently, for about five to ten minutes. In some embodiments, a composition described herein is administered twice daily for a period of two weeks or longer.

### Kits

In some embodiments, provided herein are packaged compositions, or packaged pharmaceutical compositions, and instructions for using the composition in accordance with one or more of the methods provided herein or known or approved uses of the API included in the composition. The packaged kits, in some embodiments, may comprise a container holding a therapeutically effective amount of an API in a composition described herein. In some embodiments, the kits include instructions for use.

The present compositions and associated materials can be finished as a commercial product by the usual steps performed in the present field, for example by appropriate sterilization and packaging steps. For example, the compositions and materials can be treated by UV/vis irradiation (200-500 nm), for example using photo-initiators with different absorption wavelengths (e.g., Irgacure 184, 2959), preferably water-soluble initiators (e.g., Irgacure 2959). Such irradiation is usually performed for an irradiation time of 1-60 minutes, but longer irradiation times may be applied, depending on the specific method. The compositions and materials can be finally sterile-wrapped to retain sterility until use and packaged (e.g., by the addition of specific product information leaflets) into suitable containers (e.g., boxes, bottles, bags, tubes, etc.).

According to further embodiments, the described compositions can also be provided in kit form combined with other components necessary for administration of the material to the patient. For example, disclosed kits, such as for use in the treatment of topical conditions, can further comprise, for example, administration materials.

The kits may be designed in various forms based on the specific deficiencies they are designed to treat.

The compositions provided herein may be prepared and placed in a container for storage at ambient or elevated temperature. When the compositions are stored in a polyolefin plastic container as compared to, for example, a polyvinyl chloride plastic container, discoloration of the composition or degradation of the API may be reduced. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). In some embodiments, containers also include the capacity to reduce exposure of the container's contents to infrared light, or a second component with such a capacity. In some embodiments, containers further include the capacity to reduce the exposure of the container's contents to heat or humidity. In some embodiments, containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. In some embodiments, containers may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include a composition described herein. The compositions provided herein may need storage lasting up to, or longer than, three months; in some cases up to, or longer than one year. In some embodiments, containers may be in any form suitable to contain the contents—for example, a bag, a bottle, a tube, or a box, or a combination thereof.

In some embodiments, a kit comprises a container including a non-aqueous composition (e.g., according to Example 57) comprising: about 1% w/w of tolnaftate, about 36.7% w/w of ethanol, about 30.9% w/w of polyethylene glycol, about 0.5% w/w of methylparaben, about 30% w/w of polysorbate, fragrance, and solvent to qs 100%. The kit further includes instructions to dilute the composition with water to prepare a soaking bath (e.g., for soaking hands or feet) for preventing or treating fungal infections. The kit optionally comprises an additional container including a second composition comprising: about 0.1% to about 1% w/w of undecylenic acid, about 90.5% w/w 2 ethyl cyanoacrylate, about 9% w/w polymethylmethacrylate, and about 0.4% w/w hydroquinone. The kit further includes instructions for applying the second composition as a nail paint (e.g., for preventing or treating fungal infection in the nails).

The following examples further illustrate the present disclosure. However, they are in no way a limitation of the teachings or disclosure described herein.

Always be healthy Keanu Langroudi and Seilah Langroudi.

### EXAMPLES

### Example 1: Amphotericin B Solution

Amphotericin B, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 2: Amphotericin B Solution

Amphotericin B, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 3: Amphotericin B Solution

Amphotericin B, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 4: Amphotericin B Solution

Amphotericin B, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 5: Anidulafungin Solution

Anidulafungin, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 6: Anidulafungin Solution

Anidulafungin, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 7: Anidulafungin Solution

Anidulafungin, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 8: Anidulafungin Solution

Anidulafungin, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 9: Clotrimazole Solution

Clotrimazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 10: Clotrimazole Solution

Clotrimazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 11: Clotrimazole Solution

Clotrimazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 12: Clotrimazole Solution

Clotrimazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 13: Econazole Solution

Econazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 14: Econazole Solution

Econazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 15: Econazole Solution

Econazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 16: Econazole Solution

Econazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 17: Fluconazole Solution

Fluconazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 18: Fluconazole Solution

Fluconazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 19: Fluconazole Solution

Fluconazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 20: Fluconazole Solution

Fluconazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 21: Itraconazole Solution

Itraconazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 22: Itraconazole Solution

Itraconazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 23: Itraconazole Solution

Itraconazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 24: Itraconazole Solution

Itraconazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 25: Ketoconazole Solution

Ketoconazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 26: Ketoconazole Solution

Ketoconazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 27: Ketoconazole Solution

Ketoconazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 28: Ketoconazole Solution

Ketoconazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 29: Miconazole Solution

Miconazole, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 30: Miconazole Solution

Miconazole, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 31: Miconazole Solution

Miconazole, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 32: Miconazole Solution

Miconazole, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 33: Nystatin Solution

Nystatin, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 34: Nystatin Solution

Nystatin, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 35: Nystatin Solution

Nystatin, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 36: Nystatin Solution

Nystatin, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 37: Terbinafine Solution

Terbinafine, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 38: Terbinafine Solution

Terbinafine, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 39: Terbinafine Solution

Terbinafine, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 40: Terbinafine Solution

Terbinafine, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 41: Tolnaftate Solution

Tolnaftate, SD alcohol, polyethylene glycol 300, menthol, and methyl paraben are combined, then mixed with water to a final volume of 200 mL. The solution appears clear and colorless, giving off a menthol odor. The pH is measured at 25° C., and is found to be about 6.2.

After filtration over a bed of charcoal, and through a 0.45 μm filter, the solution is clear and colorless.

### Example 42: Tolnaftate Solution

Tolnaftate, SD alcohol, polysorbate 20, menthol, and methyl paraben are combined, then mixed with water to a final volume of 200 mL. The solution appears clear and pale yellow, giving off a menthol odor. The pH is measured at 25° C., and is found to be about 6.2.

After filtration over a bed of charcoal, and through a 0.45 μm filter, the solution is clear and colorless.

### Example 43: Tolnaftate Solution

Tolnaftate, SD alcohol, polyethylene glycol 300, and methyl paraben are combined, then mixed with water to a final volume of 200 mL. The solution appears clear and colorless.

After filtration over a bed of charcoal, and through a 0.45 μm filter, the solution is clear and colorless.

### Example 44: Tolnaftate Solution

Tolnaftate, SD alcohol, polysorbate 20, and methyl paraben are combined, then mixed with water to a final volume of 200 mL. The solution appears clear and pale yellow.

After filtration over a bed of charcoal, and through a 0.45 μm filter, the solution is clear and colorless.

### Example 45: Packaged Solution

The solutions of Examples 1-44 are packaged, separately, in a glass or plastic bodied multi-dose container for topical administration.

### Example 46: Stability

The solutions of Examples 1-44 are assayed according to standard shelf-life stability protocols. The solutions are found to be stable, showing no observable signs of degradation (e.g., by visual or by analytical assay (e.g., HPLC)) of API or precipitation after two weeks and 3 months. Under these conditions, the time represents 2 years of useful life for the solutions.

### Example 47: Stability

The solutions of Examples 1-44 are assayed according to the USP <60>, <61>, or <62> antimicrobial effectiveness testing, which is a challenge test to assess microbial contamination or proliferation and typically performed on commercial products such as, but not limited to, injections, topicals, oral products, or antacids packaged in multiple-dose containers. The samples are found to meet the acceptance criteria for the test.

### Example 48: API Patch

The solutions of Examples 1-44 are prepared by incorporation into individual 1″ by 1″ patches, each patch including one API. A box is assembled including 10 identical patches per box.

### Example 49: API Patch Effectiveness

The patch of Example 48 is applied to a skin surface of a subject having a fungal infection. After daily application of a fresh patch having the same API as the previous patch, the fungal infection is reduced as observed by reduction of visual cues identifying the fungal infection.

### Example 50: API Solution Effectiveness

The solution of one of Examples 1-44 is applied to a foot skin surface of a subject having a fungal infection resulting in Athlete's foot. After daily application of the solution, the Athlete's foot infection is reduced as observed by reduction of visual cues identifying the fungal infection.

### Example 51: Treatment Using API Aqueous Solution

API is combined with a mixture—the mixture comprising about 25-60% w/w SD alcohol, about 20-35% w/w polyethylene glycol 300, about 19.8-39.5% w/w polysorbate 20, and about 0.2-0.5% w/w methyl paraben—to form a non-aqueous solution comprising about 0.5-2% w/w (e.g., 1% w/w) API. After optional filtration over a bed of charcoal and through a 0.45 μm filter the non-aqueous solution is clear and colorless.

About 10 to 15 mL of the non-aqueous solution is dispersed in about 1.5 to 2 L water to form an API aqueous solution. A patient suffering from a foot fungal infection soaks the foot in the API aqueous solution for a period of at least 15 min once or twice a day. Improvement of one or more symptoms of the foot fungal infection is observed.

### Example 52: Treatment Using API Aqueous Solution

API is combined with a mixture—the mixture comprising about 60% w/w SD alcohol, about 20% w/w polyethylene glycol 300, about 19.8% w/w polysorbate 20, and about 0.2% w/w methyl paraben—to form a non-aqueous solution comprising about 0.5-2% w/w (e.g., 1% w/w) API. After optional filtration over a bed of charcoal and through a 0.45 μm filter the non-aqueous solution is clear and colorless.

About 10 to 15 mL of the non-aqueous solution is dispersed in about 1.5 to 2 L water to form an API aqueous solution. A patient suffering from a foot fungal infection soaks the foot in the API aqueous solution for a period of at least 15 min once or twice a day. Improvement of one or more symptoms of the foot fungal infection is observed.

### Example 53: Treatment Using API Aqueous Solution

API is combined with a mixture—the mixture comprising about 25% w/w SD alcohol, about 35% w/w polyethylene glycol 300, about 39.5% w/w polysorbate 20, and about 0.5% w/w methyl paraben—to form a non-aqueous solution comprising about 0.5-2% w/w (e.g., 1% w/w) API. After optional filtration over a bed of charcoal and through a 0.45 μm filter the non-aqueous solution is clear and colorless.

About 10 to 15 mL of the non-aqueous solution is dispersed in about 1.5 to 2 L water to form an API aqueous solution. A patient suffering from a foot fungal infection soaks the foot in the API aqueous solution for a period of at least 15 min once or twice a day. Improvement of one or more symptoms of the foot fungal infection is observed.

### Example 54: Treatment Using API Aqueous Solution

API is combined with a mixture—the mixture comprising about 60% w/w SD alcohol, about 39.5% w/w polysorbate 20, and about 0.5% w/w methyl paraben—to form a non-aqueous solution comprising about 0.5-2% w/w (e.g., 1% w/w) API. After optional filtration over a bed of charcoal and through a 0.45 μm filter the non-aqueous solution is clear and colorless.

About 10 to 15 mL of the non-aqueous solution is dispersed in about 1.5 to 2 L water to form an API aqueous solution. A patient suffering from a foot fungal infection soaks the foot in the API aqueous solution for a period of at least 15 min once or twice a day. Improvement of one or more symptoms of the foot fungal infection is observed.

### Example 55: Treatment Using API Aqueous Solution

API is combined with a mixture—the mixture comprising about 60% w/w SD alcohol, about 35% w/w polyethylene glycol 300, and about 0.5% w/w methyl paraben—to form a non-aqueous solution comprising about 0.5-2% w/w (e.g., 1% w/w) API. After optional filtration over a bed of charcoal and through a 0.45 μm filter the non-aqueous solution is clear and colorless.

About 10 to 15 mL of the non-aqueous solution is dispersed in about 1.5 to 2 L water to form an API aqueous solution. A patient suffering from a foot fungal infection soaks the foot in the API aqueous solution for a period of at least 15 min once or twice a day. Improvement of one or more symptoms of the foot fungal infection is observed.

### Example 56: API Solution

In a reaction tank, tolnaftate was added to ethanol (standard denatured alcohol 40b) and stirred for 15 minutes. Polyethylene glycol 300 was added to the tank and stirring continued for 15 minutes. Methyl paraben was added to the tank and stirring continued for 15 minutes. Polysorbate 20 was added to the tank and stirring continued for 15 minutes to obtain a final solution. The w/w percentages in the final solution are shown in Table 1. All ingredients were purchased from commercial suppliers. Fragrance is added, and solvent (e.g., ethanol) may be added to qs 100%.

TABLE 1

| Ingredient | w/w % |
|---|---|
| Tolnaftate | 1 |
| Ethanol (SD alcohol 40b) | 36.7 |
| Polyethylene glycol 300 | 30.9 |
| Methylparaben | 0.5 |
| Polysorbate 20 | 30 |

The final solution was a light yellow liquid with a specific gravity of 0.97±0.1, and tested negative or below 10 colony forming units (CFU) per gram for microbes. Solutions are likewise prepared comprising amphotericin B, anidulafungine, clotrimazole, econazole, fluconazole, itraconazole, ketoconazole, miconazole, nystatin, terbinafine, or undecylenic acid as the API instead of tolnaftate.

The tolnaftate solution was characterized; results in Table 2. Similar results are observed for the solutions comprising amphotericin B, anidulafungine, clotrimazole, econazole, fluconazole, itraconazole, ketoconazole, miconazole, nystatin, or terbinafine as the API instead of tolnaftate.

TABLE 2

| Item | Specification | Test Method | Result |
|---|---|---|---|
| Form | Liquid | Organoleptic | Pass |
| Color | Light Yellow | Organoleptic | Pass |
| Fragrance/Flavor | Characteristic | Organoleptic | Pass |
| pH @ 25° C. | 7.0 ± 1.0 | SOP QA 15B | 6.81 |
| TPC | <100 CFU per gram | USP 61 | <10 CFU/g |
| Yeast & Mold | <10 CFU per gram | USP 61 | <10 CFU/g |
| Escherichia coli | Negative | USP 62 | Negative |
| Salmonella | Negative | USP 62 | Negative |
| Staph aureus | Negative | USP 62 | Negative |
| Pseudomonas | Negative | USP 62 | Negative |
| aeruginosa | | | |
| B. Cepacia | Negative | USP 60 | Negative |
| Viscosity | N/A | SOP QA 15A | N/A |
| Specific Gravity | 0.97 ± 0.1 | LAB SG | 0.98 |
| Tolnaftate | 0.7-1.3% | ACCU#: C-064 | 1.02% |

### Example 57: Repeat Insult Patch Test for Skin Irritation and Skin Sensitization

This study enrolled 50 subjects.

Criteria for inclusion: Subjects who were not currently under a doctor's care; who were free of any dermatological or systemic disorder that would interfere with the results; subjects who were free of any acute or chronic disease that would interfere with the results; subjects who completed a preliminary medical history form mandated by ALS and were in general good health; subjects who understood and signed an informed consent document; subjects who were able to cooperated with the Investigator and research staff and were willing to have test materials applied according to the protocol and complete the full course of study.

Criteria for exclusion: Subjects under 18 years of age; subjects currently under a doctor's care; subjects on medication (topical or systemic) that might mask or interfere with the test results; subjects who had a history of any acute or chronic disease that might interfere with or increase the risk associated with study participation; subjects diagnosed with chronic skin allergies; female subjects who were pregnant or nursing.

Informed consent was obtained from each subject prior to initiating the study.

Population demographics are shown in Table 3 below:

TABLE 3

| | |
|---|---|
| Number of subjects enrolled | 91 |
| Number of subjects completing study | 55 |
| Age Range | 18-64 |
| Sex | |
| Male | 9 |
| Female | 46 |
| Fitzpatrick Skin Type* | |
| 1 - always burn, does not tan | 0 |
| 2 - burn easily, tan slightly | 9 |
| 3 - burn moderately, tan progressively | 27 |
| 4 - burn a little, always tan | 19 |
| 5 - rarely burn, tan intensely | 0 |
| 6 - never burn, tan very intensely | 0 |

*Agache P., Hubert P. Measuring the skin (p. 473, table 48.1) Springer-Verlag Berlin Heidelberg, 2004

Materials: A 1% formulation of tolnaftate was tested.

Test materials to be tested under occlusive conditions were placed on an adhesive tape with paper filter discs with 1.0 cm² (Adhesive tapes from 3M Company—Durapore (Code1538) and Blenderm (Code 1525) or placed on an 8-millimeter aluminum Finn Chamber® (Epitest Ltd. Oy, Tuusula, Finland) supported on Scanpor® Tape (Norgesplaster A/S, Kristiansand, Norway) or an a-millimeter filter paper coated aluminum Finn Chamber® AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRA® closed cell system which is made of additive-free polyethylene plastic foam with a filter paper incorporated (It is supplied in units of 10 chambers on a hypoallergenic nonwoven adhesive tape; the width of the tape is 52 mm and the length is 118 mm) or other equivalents.

Test materials to be tested under semi-occlusive conditions were placed on an adhesive tape with paper filter discs with 1.0 cm² (Adhesive tapes from 3M Company
Durapore (Code 1538) or placed on a test strip with a Rayon/Polypropylene pad or on a 7.5 mm filter paper disc affixed to a strip of hypoallergenic tape (Johnson & Johnson 1 inch First Aid Cloth Tape).

Test materials to be tested in an open patch were applied and rubbed directly onto the back of the subject. Approximately 0.02-0.05 ml (in case of liquids) and/or 0.02-0.05 gm (in case of solids) of the test material was used for the study. Liquid test material was dispensed on a paper disk, which fit in the patch chamber.

### Procedure:

Subjects were requested to bathe or wash as usual before arrival at the facility.

Patches containing the test material were then affixed directly to the skin of the intrascapular regions of the back, to the right or left of the midline and subjects were dismissed with instructions not to wet or expose the test area to direct sunlight.

Approximately 0.02-0.05 ml of distilled water was applied as a negative control. This was applied on an occlusive patch at the first induction only.

Patches remained in place for 48 hours after the first application. Subjects were instructed not to remove the patches prior to their 48 hour scheduled visit. Thereafter, subjects were instructed to remove patches 24 hours after application for the remainder of the study.

This procedure was repeated until a series of eight (8) to nine (9) consecutive, 24-hour exposures had been made three (3) times a week for three (3) consecutive weeks.

Prior to each reapplication, the test sites were evaluated by trained laboratory personnel. Following a 10-14 day rest period a retest/challenge dose was applied once to a previously unexposed test site. Test sites were evaluated by trained laboratory personnel 48 and 96 hours after application.

In the event of a reaction, the area of erythema and edema were measured.

Edema is estimated by the evaluation of the skin with respect to the contour of the unaffected normal skin. Subjects were instructed to report any delayed reactions that might occur after the final reading.

Scoring: Scoring scale and definition of symbols shown below are based on the scoring scheme according to the International Contact Dermatitis Research Group scoring scale (Rietschel, R. L., Fowler, J. F., Ed., Fisher's Contact Dermatitis (fourth ed.). Baltimore, Williams & Wilkins, 1995).
0 no reaction
1 erythema throughout at least ¾ of patch area
2 erythema and induration throughout at least ¾ of patch area
3 erythema, induration and vesicles
4 erythema, induration and bullae
D Site discontinued
Dc Subject discontinued voluntarily
Dcl Subject discontinued per Investigator

Clinical evaluations were performed by an investigator or designee trained in the clinical evaluation of the skin. Whenever feasible, the same individual did the scoring of all the subjects throughout the study and was blinded to the treatment assignments and any previous scores.

Conclusion: The test product was dermatologist tested and under the conditions of the study, there was no indication of a potential to elicit dermal irritation or sensitization (contact allergy).

### Example 58: Antifungal Soak Market Surveys

Subjects who underwent manicures or pedicures were provided surveys. The data is shown in Table 4 and Table 5.

TABLE 4

| Patient | | | likely | Belief in | Pay |
|---|---|---|---|---|---|
| No. | awareness^{a} | importance^{b} | to use^{c} | health effect^{d} | more^{e} |
| 1. | 1 | 1 | 1 | 1 | 5 |
| 2. | 2 | 1 | 1 | 1 | 2 |
| 3. | 1 | 1 | 1 | 1 | 2 |
| 4. | 2 | 1 | 2 | 1 | 4 |
| 5. | 1 | 1 | 1 | 1 | 1 |
| 6. | 2 | 2 | 1 | 2 | 3 |
| 7. | 2 | 1 | 1 | 1 | 3 |
| 8. | 2 | 1 | 1 | 1 | 2 |
| 9. | 2 | 2 | 1 | 2 | 2 |
| 10. | 2 | 1 | 1 | 1 | 1 |
| 11. | 2 | 1 | 1 | 1 | — |
| 12. | 2 | 1 | 2 | 2 | 2 |
| 13. | 2 | 1 | 2 | 2 | 3 |
| 14. | 2 | 1 | 1 | 1 | 2 |
| 15. | 2 | 1 | 2 | 1 | 3 |
| 16. | 2 | 2 | 1 | 1 | 3 |
| 17. | 2 | 1 | 1 | 2 | 2 |
| 18. | 2 | 2 | 2 | 1 | 3 |
| 19. | 2 | 1 | 1 | 2 | 4 |
| 20. | 2 | 2 | 1 | 1 | 4 |
| 21. | 2 | 2 | 2 | 2 | 3 |
| 22. | 2 | 1 | 1 | 1 | 4 |
| 23. | 2 | 1 | 1 | 1 | 2 |
| 24. | 2 | 1 | 1 | 2 | 3 |

^{a}Before taking this survey, were you aware of the availability of antifungal products: specifically designed to be used during manicures and pedicures for prevention: Yes (1) - No (2)
^{b}how important is it to use preventive antifungal soaks during manicures and pedicures: very important (1) - important (2) - neutral (3) -, not important (4) - not important at all (5)
^{c}how likely are you to use preventive antifungal soaks during manicures and pedicures: very likely (1) - likely (2) - neutral (3) -, unlikely (4) - very unlikely (5)
^{d}Do you believe that using a preventative antifungal soak can improve the overall health of your nails and skin: strongly agree (1) - agree (2) - neutral (3) -, disagree (4) - strongly disagree (5)
^{e}how much more would you pay for a manicure or pedicure that includes a preventative antifungal soak: no additional cost (1) - up to 10% more (2) - 10-20% more (3) -, 20-30% more (4) - more than 30% (5)

TABLE 5

| Patient No. | attributes^{a} | using a soak^{b} | recommend to others^{c} |
|---|---|---|---|
| 1. | 1 | 1 | 1 |
| 2. | 1 | 1 | 2 |
| 3. | 2 | 2 | 2 |
| 4. | 1 | 1 | 2 |
| 5. | 1 | 1 | 1 |
| 6. | 1 | 1 | 1 |
| 7. | 2 | 1 | 1 |
| 8. | 2 | 1 | 1 |
| 9. | 1 | 2 | 2 |
| 10. | 1 | 2 | 2 |
| 11. | — | 1 | 1 |
| 12. | 1 | 1 | 1 |
| 13. | 1 | 1 | 1 |
| 14. | 2 | 1 | 1 |
| 15. | 1 | 2 | 1 |
| 16. | 2 | 2 | 2 |
| 17. | 1 | 1 | 1 |
| 18. | 1 | 1 | 1 |
| 19. | 1 | 1 | 1 |
| 20. | 1 | 1 | 1 |
| 21. | 1 | 1 | 2 |
| 22. | 2 | 1 | 1 |
| 23. | 2 | 1 | 1 |
| 24. | 1 | 1 | 1 |

^{a}How important are the following attributes in a preventative antifungal soak for manicures and pedicures? Efficacy in preventing fungal infections - Ease of use - Pleasant fragrance - Natural ingredients - Price: very important (1) - important (2) - neutral (3) -, not important (4) - not important at all (5)
^{b}How do you feel about the idea of using a preventative antifungal soak during your manicures and pedicures: very positive (1) - positive (2) - neutral (3) -, negative (4) - very negative (5)
^{c}Would you recommend using a preventative antifungal soak to others who get manicures and pedicures: definitely (1) - probably (2) - not sure (3) -, probably not (4)

The data in Table 4 and Table 5 shows that subjects are receptive to the idea of an antifungal soak being available in the market and that there is a market need for such a product.

### Example 59: Post Treatment Evaluation

Subjects who had previously tried anti-fungal creams were treated with an antifungal soak described herein during a manicure or a pedicure. One ounce of a tolnaftate composition described herein was diluted in one gallon of water and used as a soak solution during a manicure or a pedicure.

After the treatment regimen, patients filled out surveys. The data from the surveys is shown in Table 6 and Table 7.

TABLE 6

| Patient | Ease | Overall | | Overall | Recommend |
|---|---|---|---|---|---|
| No. | of use^{a} | comfort^{b} | Fragrance^{cc} | satisfaction^{d} | to others^{e} |
| 1. | 2 | 2 | 2 | 2 | 1 |
| 2. | 1 | 1 | 1 | 1 | 1 |
| 3. | 1 | 1 | 2 | 1 | 1 |
| 4. | 1 | 1 | 1 | 1 | 1 |
| 5. | 1 | 1 | 1 | 1 | 1 |
| 6. | 2 | 2 | 1 | 3 | 2 |
| 7. | 1 | 2 | 3 | 1 | 2 |
| 8. | 1 | 2 | 3 | 1 | 1 |
| 9. | 2 | 2 | 3 | 2 | 2 |
| 10. | 2 | 1 | 2 | 1 | 2 |
| 11. | 1 | 2 | 3 | 2 | 1 |
| 12. | 2 | 1 | 1 | 1 | 1 |
| 13. | 1 | 2 | 3 | 1 | 2 |
| 14. | 2 | 1 | 3 | 1 | 2 |
| 15. | 1 | 1 | 3 | 1 | 1 |
| 16. | 2 | 2 | 3 | 1 | 1 |
| 17. | 2 | 2 | 3 | 1 | 2 |
| 18. | 2 | 1 | 1 | 1 | 1 |
| 19. | 1 | 1 | 3 | 1 | 1 |
| 20. | 1 | 1 | 1 | 1 | 1 |
| 21. | 2 | 2 | 2 | 1 | 2 |
| 22. | 1 | 1 | 3 | 2 | 2 |
| 23. | 2 | 2 | 3 | 1 | 1 |
| 24. | 2 | 2 | 1 | 2 | 2 |
| 25. | 2 | 2 | 2 | 1 | 2 |
| 26. | 2 | 2 | 3 | 1 | 2 |
| 27. | 2 | 1 | 2 | 1 | 1 |
| 28. | 1 | 2 | 1 | 2 | 1 |

^{a}ease of use compared to antifungal creams: Much easier (1) - Easier (2) - About the same (3) -, Harder (4) - Much harder (5)
^{b}overall comfort of using an antifungal soak compared to antifungal creams: very comfortable (1) - comfortable (2) - neutral (3) -, uncomfortable (4) - very uncomfortable (5)
^{c}fragrance of antifungal soak very pleasant (1) - pleasant (2) - neutral (3) -, unpleasant (4) - very unpleasant (5)
^{d}overall satisfaction using an antifungal soak compared to antifungal creams: very satisfied (1) - satisfied (2) - neutral (3) -, unsatisfied (4) - very unsatisfied (5)
^{e}how likely to recommend antifungal soak compared to antifungal creams to other people definitely (1) - probably (2) - not sure (3) -, probably not (4) - definitely not (5)

TABLE 7

| | Effectiveness of antifungal | Duration of relief provided |
|---|---|---|
| Patient | soak compared to antifungal | by antifungal soak compared |
| No. | creams^{a} | to antifungal creams^{b} |
| 1. | — | 3 |
| 2. | 1 | 1 |
| 3. | 2 | 1 |
| 4. | 2 | 1 |
| 5. | 1 | 1 |
| 6. | 1 | 1 |
| 7. | 1 | 1 |
| 8. | 2 | 1 |
| 9. | 3 | 1 |
| 10. | 1 | 1 |
| 11. | 2 | 1 |
| 12. | 1 | 1 |
| 13. | 2 | 1 |
| 14. | 1 | 1 |
| 15. | 2 | 1 |
| 16. | 2 | 1 |
| 17. | 2 | 1 |
| 18. | 1 | 1 |
| 19. | 1 | 1 |
| 20. | 2 | 2 |
| 21. | 1 | 1 |
| 22. | 1 | 2 |
| 23. | 2 | 2 |
| 24. | 2 | 1 |
| 25. | 2 | 1 |
| 26. | 2 | 1 |
| 27. | 1 | 1 |
| 28 | 3 | 1 |

— Indicates no response from patient
^{a}Much more effective (1) - More effective (2) - Equally effective (3) - Less effective (4) - Much less effective (5)
^{b}Very satisfied (1) - Satisfied (2) - Neutral (3) - Dissatisfied (4) - Very dissatisfied (5)

The data in Table 6 shows that subjects regard an antifungal soak favorably. The data in Table 7 shows that two patients rated the soak as equal to antifungal creams, while all the remaining patients rated the antifungal soak as better than antifungal creams. All patients were satisfied with the duration of relief provided by the antifungal soak compared to antifungal creams. Thus, the methods described herein are an improvement over antifungal creams.

We claim:

1. A composition comprising about 0.5 to about 2% w/w of an active pharmaceutical ingredient selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin; and
a mixture comprising about 25% to about 60% w/w of a C₁₋₄ alcohol and about 19% to about 75% w/w of at least one nonionic surfactant.

2. The composition of claim 1, wherein the C₁₋₄ alcohol is ethanol.

3. The composition of claim 1, wherein the nonionic surfactant is selected from a polyethylene glycol, polysorbate 20, or a combination thereof.

4. The composition of claim 1, wherein:
the C₁₋₄ alcohol is ethanol; and
the at least one nonionic surfactant is polyethylene glycol 300 or polysorbate 20, or a combination thereof.

5. The composition of claim 1, comprising about 1% w/w of the active pharmaceutical ingredient.

6. The composition of any one of claims 1-5, the mixture comprising:
about 25% w/w of the C₁₋₄ alcohol;
about 74.5% w/w of the at least one nonionic surfactant; and
about 0.5% w/w of a preservative.

7. The composition of any one of claims 1-5, the mixture comprising:
about 60% w/w of the C₁₋₄ alcohol;
about 39.8% w/w of the at least one nonionic surfactant; and
about 0.2% w/w of a preservative.

8. The composition of any one of claims 1-5, the mixture comprising:
about 37% w/w of the C₁₋₄ alcohol;
about 61% w/w of the at least one nonionic surfactant; and
about 0.5% w/w of a preservative.

9. The composition of claim 1, the mixture comprising:
about 25-60% w/w ethanol;
about 20-35% w/w polyethylene glycol 300;
about 19.8-39.5% w/w polysorbate 20; and
about 0.2-0.5% w/w methyl paraben.

10. The composition of claim 1, the mixture comprising:
A) about 60% w/w ethanol;
about 20% w/w polyethylene glycol 300;
about 19.8% w/w polysorbate 20; and
about 0.2% w/w methyl paraben; or
B) about 25% w/w ethanol;
about 35% w/w polyethylene glycol 300;
about 39.5% w/w polysorbate 20; and
about 0.5% w/w methyl paraben; or
C) about 60% w/w ethanol;
about 39.5% w/w polysorbate 20; and
about 0.5% w/w methyl paraben; or
D) about 60% w/w ethanol;
about 35% w/w polyethylene glycol 300; and
about 0.5% w/w methyl paraben; or
E) about 37% w/w ethanol;
about 30% w/w polysorbate 20; and
about 0.5% w/w methyl paraben; or
F) about 37% w/w ethanol;
about 31% w/w polyethylene glycol 300; and
about 0.5% w/w methyl paraben.

11. The composition of claim 1, comprising a mixture comprising:
about 0.5 to about 1.5% w/w of an active pharmaceutical ingredient having a water solubility of less than about 1 mg/mL;
about 37% w/w ethanol;
about 31% w/w polyethylene glycol 300;
about 30% w/w polysorbate 20; and
about 0.5% w/w of methylparaben;
and optionally further comprising at least one odorant up to about 0.5% w/w, and optionally further comprising one or more of caspofungin or micafungin up to about 0.5 or 1% w/w;
and optionally wherein the mixture is diluted from about 1:1 to 1:150 in water.

12. The composition of any one of claims 1-11, further comprising 0 to about 5% w/w of one or more odorant.

13. A composition, comprising a C₁₋₄ alcohol, at least one nonionic surfactant, and an active pharmaceutical ingredient, wherein:
the composition is an aqueous composition;
the active pharmaceutical ingredient is dissolved in the aqueous composition; and
the active pharmaceutical ingredient has a water solubility of less than 1 mg/mL.

14. The composition of claim 13, the composition comprising about 0.5 to about 2% w/w of the active pharmaceutical ingredient, water, and a mixture, the mixture comprising:
about 25% to about 60% w/w of the C₁₋₄ alcohol; and
about 19% to about 75% w/w of the at least one nonionic surfactant.

15. The composition of claim 13 or 14, wherein:
the C₁₋₄ alcohol is ethanol;
the at least one nonionic surfactant is selected from polyethylene glycol 300 or polysorbate 20, or a combination thereof; and
the active pharmaceutical ingredient is selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin.

16. The composition of claim 13, wherein the C₁₋₄ alcohol is ethanol.

17. The composition of claim 13 or 14, wherein the at least one nonionic surfactant is selected from a polyethylene glycol, polysorbate 20, or a combination thereof.

18. The composition of one of claims 13-17, wherein the at least one nonionic surfactant includes polyethylene glycol 300.

19. The composition of one of claims 13-16, further comprising a preservative.

20. The composition of claim 14, the mixture further comprising about 0.2% to about 0.5% w/w of a preservative.

21. The composition of claim 19 or 20, wherein the preservative is a C₁₋₄ alkyl ester of para-hydroxybenzoic acid.

22. The composition of one of claims 19-21, wherein the preservative is methyl 4-hydroxybenzoate.

23. The composition of one of claims 13-22, further comprising at least one odorant.

24. The composition of one of claims 13-22, further comprising at least one monoterpene or monoterpenoid.

25. The composition of one of claims 13-22, further comprising menthol, limonene, carvone, hinokitiol, or linalool.

26. The composition of claim 13, comprising:
ethanol;
a nonionic surfactant selected from polyethylene glycol 300 or polysorbate 20, or a combination thereof;
menthol, limonene, carvone, hinokitiol, or linalool; and
methyl 4-hydroxybenzoate;
wherein the active pharmaceutical ingredient is selected from one or more of amphotericin B, anidulafungine, clotrimazole, econazole, fluconazole, itraconazole, ketoconazole, miconazole, nystatin, terbinafine, or tolnaftate, optionally further including one or more of caspofungin or micafungin.

27. The composition of claim 13, comprising about 1% w/w of the active pharmaceutical ingredient, water, and a mixture, and optionally further comprising 0 to about 1% w/w of at least one odorant,
wherein the mixture comprises:
about 25% w/w of the C₁₋₄ alcohol;
about 74.5% w/w of the nonionic surfactant; and
about 0.5% w/w of a preservative.

28. The composition of claim 13, comprising about 1% w/w of the active pharmaceutical ingredient, water, and a mixture, and optionally further comprising 0 to about 1% w/w of at least one odorant,
wherein the mixture comprises:
about 60% w/w of the C₁₋₄ alcohol;
about 39.8% w/w of the nonionic surfactant; and
about 0.2% w/w of a preservative.

29. The composition of claim 13, comprising about 1% w/w of the active pharmaceutical ingredient, water, and a mixture, and optionally further comprising 0 to about 1% w/w of at least one odorant,
wherein the mixture comprises:
about 37% w/w of the C₁₋₄ alcohol;
about 61% w/w of the nonionic surfactant; and
about 0.5% w/w of a preservative.

30. The composition of one of claims 1-29, which does not comprise pulegone, myrcene, or both.

31. The composition of one of claims 1-30, further comprising undecylenic acid.

32. The composition of one of claims 1-31, further comprising hydrocortisone.

33. The composition of one of claims 1-33, prepared by a method comprising combining the components without water to prepare a mixture thereof, followed by combining the mixture with water such that the active pharmaceutical ingredient is dissolved and forms the aqueous composition.

34. A method, comprising administration of the composition of one of claims 1-33 to a subject in need thereof.

35. A method of treating a disease or condition in a subject in need thereof, comprising administration of the composition of one of claims 1-33 to the subject, wherein the disease or condition is one that the active pharmaceutical ingredient is approved for treating.

36. A method of treating a fungal condition in a subject in need thereof, comprising administration of the composition of one of claims 1-33 to the subject, wherein the fungal condition is a topical fungal condition.

37. The method of one of claims 34-36, wherein the administration is topical administration.
